(19) United States
(12) Patent Application Publication
(10) Pub. No.: US 2025/0025132 A1
(43) Pub. Date: Jan. 23, 2025

(54) ULTRASOUND DIAGNOSTIC APPARATUS, NON-TRANSITORY COMPUTER-READABLE RECORDING MEDIUM STORING ULTRASOUND DIAGNOSTIC PROGRAM, AND INFORMATION DISPLAY METHOD OF ULTRASOUND DIAGNOSTIC APPARATUS

(71) Applicant: Konica Minolta, Inc., Tokyo (JP)

(72) Inventor: Yoshihiro TAKEDA, Tokyo (JP)

(21) Appl. No.: 18/776,420

(22) Filed: Jul. 18, 2024

(30) Foreign Application Priority Data
Jul. 20, 2023 (JP) 2023-118462

(51) Int. Cl. A61B 8/00; A61B 8/08; A61B 8/14

(52) U.S. Cl. CPC A61B 8/461; A61B 8/14; A61B 8/485; A61B 8/488

(57) ABSTRACT

Provided is an ultrasound diagnostic apparatus including: an ultrasound probe that transmits and receives ultrasound to and from a subject; a display; and at least one hardware processor. The at least one hardware processor generates ultrasound image data based on a reception signal obtained from an observation area, estimates a measurement item candidate that is measurable or unmeasurable, and causes the display to display the measurement item candidate.

### CROSS REFERENCE TO RELATED APPLICATIONS

The present invention claims priority under 35 U.S.C. § 119 to Japanese Patent Application No. 2023-118462, filed on Jul. 20, 2023, the entire content of which is incorporated herein by reference.

### BACKGROUND

### Technological Field

The present invention relates to an ultrasound diagnostic apparatus, a non-transitory computer-readable recording medium storing an ultrasound diagnostic program, and an information display method of an ultrasound diagnostic apparatus.

### Description of Related Art

There is known an ultrasound diagnostic apparatus that transmits and receives ultrasound to and from a subject such as a living body by using an ultrasound probe and generates ultrasound image data for diagnosis based on a signal obtained from an ultrasound echo due to the reflection of the ultrasound.

In recent years, ultrasound diagnostic apparatuses have used an artificial intelligence (AI) technology to automatically recognize a target organ or tissue from ultrasound image data and perform automatic measurement by using a recognition result, and the AI technology has been used for various purposes.

For example, Japanese Patent Publication Laid-Open No. 2022-47609 describes that a measurement condition candidate(s) for ultrasound image data is estimated by using an AI technology, a measurement condition is determined from the estimated measurement condition candidate(s), and measurement for the ultrasound image data is performed based on the determined measurement condition.

Further, Japanese Patent Publication Laid-Open No. 2019-154654 describes that it is determined by using an AI technology whether a cross-sectional image obtained by using ultrasound is an image of a target cross section, and in a case where it is determined that the cross-sectional image is an image of the target cross section, predetermined measurement processing is executed on the determined image of the target cross section.

In Japanese Patent Publication Laid-Open No. 2022-47609 and Japanese Patent Publication Laid-Open No. 2019-154654, a measurement condition candidate(s) is/are estimated by using an AI technology, a measurement condition is determined, and it is determined that a cross-sectional image is an image of a target cross section, and thus, measurement is automatically executed by using the determined measurement condition and the determined cross-sectional image, and a measurement result is displayed.

In a case where there is a plurality of measurement items for a region displayed in a cross-sectional image, however, measurement different from the measurement item desired by a user may be executed. For example, in a case where the long diameter of the abdomen of a fetus is desired to be measured using a cross-sectional image of the abdomen of the fetus, a circumferential length thereof may be measured.

### SUMMARY

An object of the present invention is to provide an ultrasound diagnostic apparatus, a non-transitory computer-readable recording medium storing an ultrasound diagnostic program, and an information display method of an ultrasound diagnostic apparatus, each capable of executing a measurement item desired by a user.

In order to achieve at least one of the above-described objects, an ultrasound diagnostic apparatus reflecting one aspect of the present invention includes: an ultrasound probe that transmits and receives ultrasound to and from a subject; a display; and at least one hardware processor, and the at least one hardware processor generates ultrasound image data based on a reception signal obtained from an observation area, estimates a measurement item candidate that is measurable or unmeasurable, and causes the display to display the measurement item candidate.

In order to achieve at least one of the above-described objects, an ultrasound diagnostic apparatus reflecting one aspect of the present invention includes: an ultrasound probe that transmits and receives ultrasound to and from a subject; a display; and at least one hardware processor, and the at least one hardware processor generates ultrasound image data based on a reception signal obtained from an observation area, designates a measurement item, estimates whether the measurement item having been designated is measurable, and causes the display to display information on whether the measurement item having been designated is measurable.

In order to achieve at least one of the above-described objects, a non-transitory computer-readable recording medium storing an ultrasound diagnostic program reflecting one aspect of the present invention is a non-transitory computer-readable recording medium storing an ultrasound image processing program that causes a computer to execute: transmitting and receiving, by an ultrasound probe, ultrasound to and from a subject; generating ultrasound image data based on a reception signal obtained from an observation area; estimating a measurement item candidate that is measurable or unmeasurable; and displaying the measurement item candidate.

In order to achieve at least one of the above-described objects, an information display method of an ultrasound diagnostic apparatus reflecting one aspect of the present invention includes: transmitting and receiving, by an ultrasound probe, ultrasound to and from a subject; generating ultrasound image data based on a reception signal obtained from an observation area, estimating a measurement item candidate that is measurable or unmeasurable; and displaying, by a display, the measurement item candidate.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating an example of a main part of an ultrasound diagnostic apparatus according to an embodiment of the present invention,

FIG. 2 is a block diagram illustrating an example of an information display method of the ultrasound diagnostic apparatus illustrated in FIG. 1,

FIG. 3 is a diagram illustrating an example of an AI technology used in a measurement item estimation unit of the ultrasound diagnostic apparatus illustrated in FIG. 1,

FIG. 4 is a diagram illustrating an example of a learning data set used in the AI technology illustrated in FIG. 3,

FIG. 5 is a diagram illustrating an exemplary display image and exemplary measurement item candidates displayed on a display unit of the ultrasound diagnostic apparatus illustrated in FIG. 1,

FIG. 6 is a diagram illustrating another exemplary display image and other exemplary measurement item candidates displayed on the display unit of the ultrasound diagnostic apparatus illustrated in FIG. 1,

FIG. 7 is a diagram illustrating yet another exemplary display image and yet other exemplary measurement item candidates displayed on the display unit of the ultrasound diagnostic apparatus illustrated in FIG. 1,

FIG. 8 is a diagram illustrating still another exemplary display image and still another measurement item candidate displayed on the display unit of the ultrasound diagnostic apparatus illustrated in FIG. 1,

FIG. 9 is a block diagram illustrating another example (Variation 1) of the main part of the ultrasound diagnostic apparatus according to the embodiment of the present invention; and

FIG. 10 is a block diagram provided for describing an example of an information display method of the ultrasound diagnostic apparatus illustrated in FIG. 9.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present invention will be described in detail with reference to the accompanying drawings.

### [Ultrasound Diagnostic Apparatus]

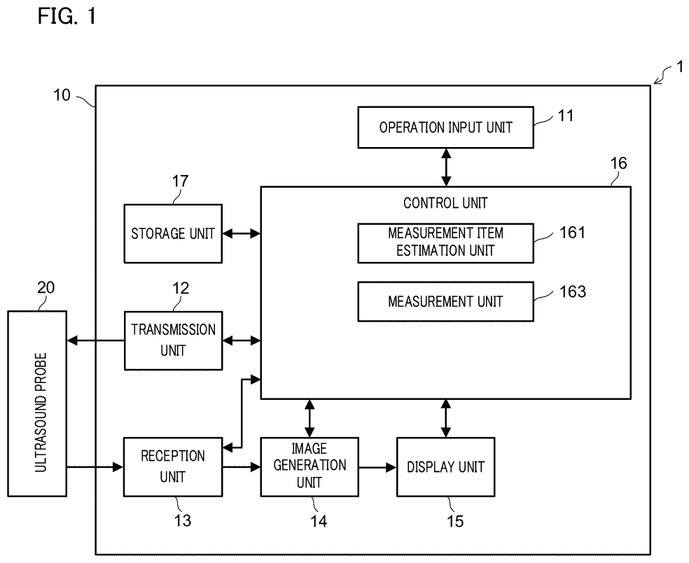

An ultrasound diagnostic apparatus according to the present embodiment will be described with reference to FIG. 1. FIG. 1 is a block diagram illustrating an example of a main part of ultrasound diagnostic apparatus 1 according to the present embodiment.

Ultrasound diagnostic apparatus 1 is used for visualizing, as an ultrasound image, the shape, properties, or dynamics of a biological tissue inside a subject such as a living body, and diagnosing the image.

As illustrated in FIG. 1, ultrasound diagnostic apparatus 1 includes apparatus main body 10, ultrasound probe (the ultrasound probe in the present invention) 20, and the like. In ultrasound diagnostic apparatus 1, ultrasound probe 20 is connected to apparatus main body 10 via a cable but may be connected to apparatus main body 10 via wireless communication.

### [Apparatus Main Body]

As illustrated in FIG. 1, apparatus main body 10 includes operation input unit 11, transmission unit 12, reception unit 13, image generation unit 14, display unit (display) 15, control unit 16, storage unit 17, and the like.

Operation input unit 11 is a user interface for a user (e.g., a healthcare professional such as a doctor or a laboratory technician) to perform an input operation, converts an input operation performed by the user into an operation signal, and inputs the operation signal to control unit 16. Operation input unit 11 is constituted by, for example, a control panel including a plurality of keys (buttons), a keyboard, a mouse, and the like. In a case where a touch screen display is used as display unit 15, the touch screen portion functions as a part of operation input unit 11 and detects, for example, a touch input for selecting a measurement item to be described later.

With operation input unit 11, the user performs, for example, an input operation such as an operation at the time of measurement by ultrasound diagnostic apparatus 1, an operation at the time of diagnosis by ultrasound diagnostic apparatus 1, and an operation to information to be displayed on display unit 15.

Transmission unit 12 is a transmission circuit that generates a voltage pulse that is a driving signal, and outputs the voltage pulse to each acoustic element of ultrasound probe 20. Transmission unit 12 sets the voltage amplitude, pulse width, and timing of a voltage pulse for each channel (each acoustic element).

For example, transmission unit 12 changes a delay time of a voltage pulse to be supplied to each acoustic element such that ultrasound outputted from each acoustic element is focused on a focal point set within a subject (delay processing).

In addition, transmission unit 12 scans the inside of a subject with ultrasound by sequentially driving the acoustic elements by channel switching.

Reception unit 13 is a reception circuit that receives and processes a reception signal generated by each acoustic element by reception of an ultrasound echo due to the reflection of ultrasound.

Reception unit 13 amplifies and A/D converts the reception signal of each channel, and provides a delay time to the reception signal after the A/D conversion. At this time, reception unit 13 gives a delay time such that the phases of reception signals generated by ultrasound echoes from a focal point set within a subject are aligned (delay processing). As a result, reception unit 13 combines reception signals of a plurality of channels into one (hereinafter, referred to as “DAS (Delay-and-Sum) signal”). Then, reception unit 13 performs filter processing on the DAS signal and outputs the DAS signal to image generation unit 14.

Image generation unit 14 performs processing of generating image data of an ultrasound image (hereinafter referred to as ultrasound image data) serving as two-dimensional frame data, based on the DAS signal (reception data) outputted from reception unit 13. Image generation unit 14 generates, for example, ultrasound image data such as a B-mode image, a Doppler image (such as a color Doppler image, a pulse Doppler image, a continuous wave Doppler image, and a tissue Doppler image), an M-mode image, and an elastography image, according to the scanning mode. The measurement item to be described later is related to an observation area of these image data.

Image generation unit 14 generates ultrasound image data after performing, for example, logarithmic compression processing, detection processing, FFT analysis processing, and the like. Since the processing of generating ultrasound image data in image generation unit 14 may be the same as processing known in the art, a description thereof will be omitted herein. Further, the generated ultrasound image data may be stored in, for example, storage unit 17.

Further, image generation unit 14 converts ultrasound image data generated by image generation unit 14 into a display signal corresponding to display unit 15, and outputs the display signal as a display image. Further, image generation unit 14 also converts a measurement item candidate, which will be described later, into a display signal corresponding to display unit 15, and outputs the display signal.

Display unit 15 is constituted by, for example, a liquid crystal display, an organic EL display, a CRT display, a touch screen display, or the like. Display unit 15 displays a display image inputted from image generation unit 14 as an ultrasound image. Note that display unit 15 is not limited to a display unit fixed to apparatus main body 10, and may be an external display connected to apparatus main body 10 by wire, a tablet wirelessly connected to apparatus main body 10, or the like.

Control unit 16 is a so-called computer, and includes, albeit illustration is omitted, a central processing unit (CPU) as an arithmetic/control apparatus, a read only memory (ROM) and a random access memory (RAM) as main storage apparatuses, and the like. Control unit 16 includes at least one hardware processor.

Programs and setting data are stored in a non-transitory computer-readable recording medium, and programs and setting data are stored in the ROM from the recording medium. The CPU reads a program according to processing contents from the ROM, develops the program in the RAM, and executes the developed program to centrally control an operation of each functional block of ultrasound diagnostic apparatus 1. That is, control unit 16 controls ultrasound diagnostic apparatus 1 in its entirety by controlling operation input unit 11, transmission unit 12, reception unit 13, image generation unit 14, and display unit 15 according to functions thereof.

In the present embodiment, control unit 16 includes measurement item estimation unit 161 and measurement unit (measurer) 163 in addition to a control block for controlling each unit.

Measurement item estimation unit 161 estimates, for example, based on reception data from reception unit 13, a measurement item candidate(s) that is/are measurable or unmeasurable. Here, measurement items that become measurement item candidates are items of measurement in an observation area of a subject and include the area and length of the observation area, and also include, for example, a circumferential long diameter, an imaged region, an imaging range, periodicity of movement of an imaging target, and the like. Details of measurement item estimation unit 161 will be described later.

Measurement unit 163 measures a measurement item of an observation area of a subject. Measurement unit 163 includes measurement processing (e.g., a measurement program) according to each measurement item. Measurement unit 163 has an automatic measurement mode and a manual measurement mode. In a case where measurement item estimation unit 161 can estimate a measurable measurement item candidate, measurement unit 163 operates in the automatic measurement mode, and executes, for example, measurement of a selected measurement item.

In a case where measurement item estimation unit 161 cannot estimate a measurable measurement item candidate or in a case where measurement item estimation unit 161 can estimate only an unmeasurable measurement item candidate, on the other hand, the mode of measurement unit 163 is switched to the manual measurement mode by, for example, a user operation. In this case, when the automatic measurement mode continues, measurement itself of an unmeasurable measurement item may be possible, but the accuracy thereof may be low, and the measurement accuracy may not be guaranteed. For this reason, the mode is switched to the manual measurement mode and the user designates a measurement item in an observation area, and thus, measurement unit 163 executes measurement of the designated measurement item. In this case, for example, it is possible to improve the accuracy of measurement by the user designating a region of a measurement target.

Storage unit 17 is, for example, a storage apparatus such as a hard disk drive (HDD), a solid state drive (SSD), or a flash memory, and stores ultrasound image data generated by image generation unit 14 and a measurement result measured by measurement unit 163.

Note that, transmission unit 12, reception unit 13, image generation unit 14, measurement item estimation unit 161, and measurement unit 163 as described above may be constituted by dedicated or general-purpose hardware (electronic circuit) supporting each processing, and implement each function in cooperation with control unit 16.

For example, transmission unit 12, reception unit 13, image generation unit 14, measurement item estimation unit 161, and measurement unit 163 are constituted by hardware such as an application specific integrated circuit (ASIC), a digital signal processor (DSP), or a programmable logic device (PLD). The PLD includes a field programmable gate array (FPGA) or the like. Transmission unit 12, reception unit 13, image generation unit 14, measurement item estimation unit 161, and measurement unit 163, when they are constituted by a DSP, a PLD, or the like, include processing programs thereof.

In addition, transmission unit 12, reception unit 13, image generation unit 14, measurement item estimation unit 161, and measurement unit 163 may be configured to cause a CPU or a graphics processing unit (GPU) to execute processing in each unit. In this case, the CPU and the GPU include processing programs for performing processing in each unit.

### [Ultrasound Probe]

Ultrasound probe 20 is, for example, a convex probe, a linear probe, a sector probe, a three-dimensional probe, or the like. Although illustration is omitted, ultrasound probe 20 includes a plurality of acoustic elements, or the like. Each acoustic element is, for example, a piezoelectric element capable of converting an electrical signal into a mechanical vibration and converting a mechanical vibration into an electrical signal, and capable of transmitting and receiving ultrasound.

Ultrasound probe 20 converts a voltage pulse outputted from transmission unit 12 into ultrasound, transmits the ultrasound into a subject, receives an ultrasound echo reflected by the subject, and outputs the received reception signal to reception unit 13. At this time, as described above, ultrasound probe 20 scans the inside of the subject with ultrasound by sequentially driving the acoustic elements by channel switching. Then, image generation unit 14 generates, based on the received reception signal, ultrasound image data serving as two-dimensional frame data.

### [Information Display Method of Ultrasound Diagnostic Apparatus]

Ultrasound diagnostic apparatus 1 has the configuration described above. When the user brings ultrasound probe 20 into contact with a surface (epidermis) of a subject, ultrasound diagnostic apparatus 1 acquires ultrasound image data within the subject by ultrasound probe 20, transmission unit 12, and reception unit 13. Then, ultrasound diagnostic apparatus 1 causes image generation unit 14 to generate, based on the acquired ultrasound image data, a display image, for example, a cross-sectional image, and causes display unit 15 to display the display image as an ultrasound image.

The ultrasound image data is generated as two-dimensional frame data, and real-time display is possible by displaying display images corresponding to the frame data in time series in real time. In addition, ultrasound diagnostic apparatus 1 is capable of measuring, for example, the long diameter, circumferential length, and the like of the abdomen of a fetus by performing measurement with respect to the reception data inputted from reception unit 13, the ultrasound image data and/or display image generated by image generation unit 14, and the like according to an operation of the user.

However, for example, in a case where there is a plurality of measurement items with respect to a region displayed on a display image such as a cross-sectional image, measurement different from a measurement item desired by the user may be executed.

Accordingly, in the present embodiment, measurement item estimation unit 161 in control unit 16 estimates a measurement item candidate(s) that is/are measurable or unmeasurable in an observation area, and display unit 15 displays the measurement item candidate(s) with respect to the observation area. Since display unit 15 displays measurement item candidates with respect to an observation area, the user can select a desired measurement item from the measurement item candidates, and control unit 16 can measure the selected desired measurement item by using measurement unit 163.

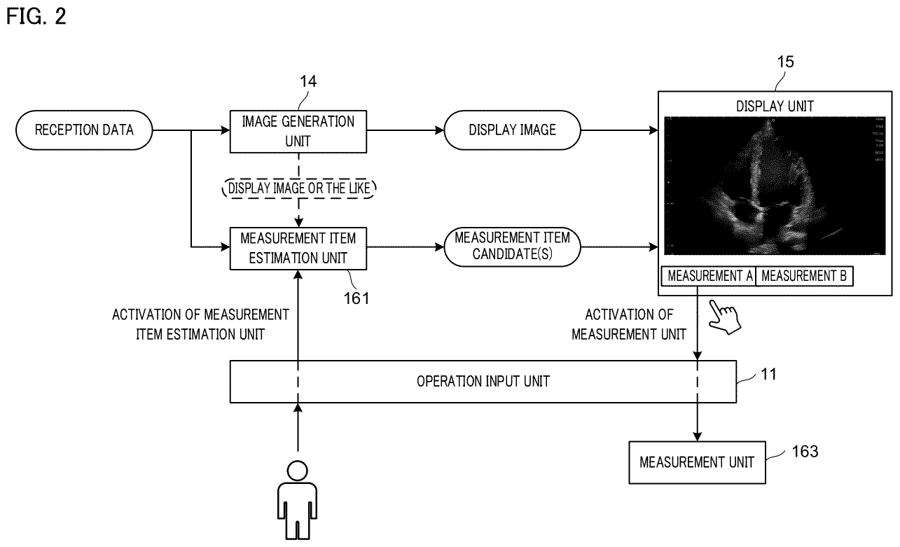

Such display and selection of a measurement item candidate(s), and measurement of the selected measurement item(s) will be described with reference to FIG. 2. FIG. 2 is a block diagram illustrating an example of an information display method of ultrasound diagnostic apparatus 1.

In ultrasound diagnostic apparatus 1, reception data acquired by using ultrasound probe 20, transmission unit 12, and reception unit 13 is inputted to image generation unit 14. The inputted reception data is generated into ultrasound image data or a display image by image generation unit 14 and is outputted as a display image, for example, a cross-sectional image to display unit 15, and display unit 15 displays the display image in real time.

At this time, measurement item estimation unit 161, when activated by an operation of the user, estimates a measurement item candidate(s), which is measurable or unmeasurable, by using the reception data from reception unit 13 as input data. Since the processing here is performed in real time according to a display image, it is desirable to estimate a measurement item candidate(s) by using the reception data. Note that, the measurement item candidate(s) may be estimated by using ultrasound image data and/or a display image generated by image generation unit 14, depending on the processing capability of control unit 16 (in a case where the processing capability thereof is high). The measurement item candidate(s) estimated by measurement item estimation unit 161 is/are outputted to display unit 15, and display unit 15 displays the measurement item candidate(s) in real time.

Display unit 15 also displays a measurement item candidate(s) together with a display image in real time. For example, as illustrated in FIG. 2, it is assumed that display unit 15 displays three of “measurement A”, “measurement B”, and “measurement C” as measurement item candidates. When the user selects a desired measurement item from “measurement A”, “measurement B”, and “measurement C”, measurement unit 163 is activated, and measurement of the selected measurement item is executed. For example, when the user selects “measurement A”, measurement unit 163 is activated, and measurement of the selected “measurement A” is executed.

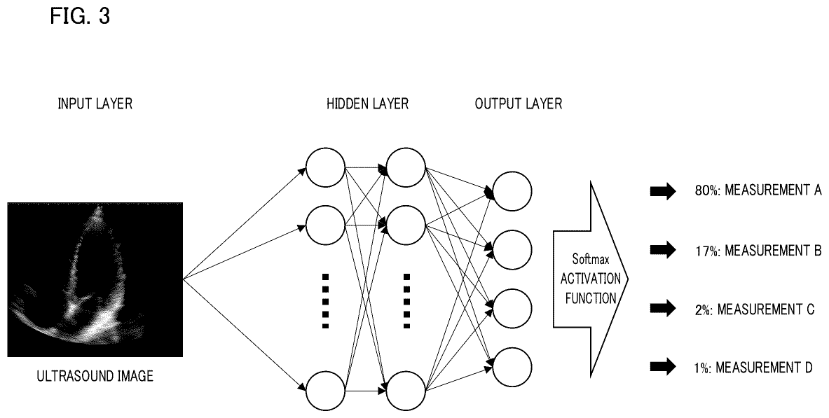

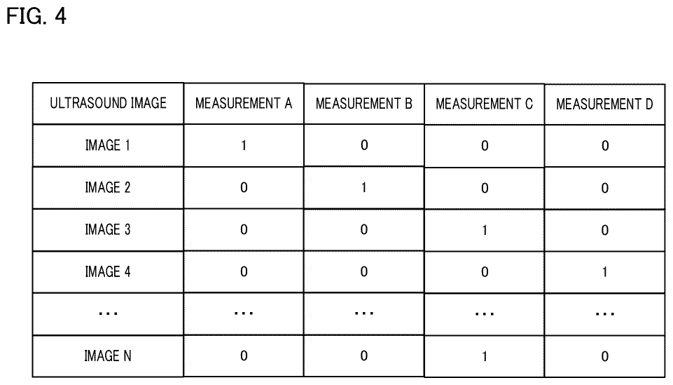

Here, the estimation in measurement item estimation unit 161 will be described with reference to FIG. 3 and FIG. 4. FIG. 3 is a diagram illustrating an example of an AI technology used in measurement item estimation unit 161 of ultrasound diagnostic apparatus 1. FIG. 4 illustrates an example of a learning data set used in the AI technology illustrated in FIG. 3.

Measurement item estimation unit 161 performs estimation using an AI technology. Measurement item estimation unit 161 is, for example, a learned model or a discriminator using a neural network, and the learned model or the discriminator is, for example, a model learned by associating an ultrasound image (cross-sectional image) with a measurement item candidate (see FIG. 3 and FIG. 4).

Note that, the learned model here is a model learned by associating an ultrasound image (cross-sectional image) with a measurement item candidate, but may be a model learned by associating reception data or ultrasound image data with a measurement item candidate.

In the present embodiment, the neural network includes an input layer, a hidden layer, an output layer, and a Softmax activation function.

The input layer is, for example, a layer that propagates information such as an ultrasound image to the hidden layer. The hidden layer is a layer that extracts a feature of information. The output layer is a layer that determines the probability of a label to be classified. The Softmax activation function converts output values of labels into values which are in a range from 0 (0%) to 1 (100%) and in which the sum of the output values of all the labels is 1 (100%).

In the neural network, the weights and thresholds of neurons of the hidden layer and the output layer are adjusted by learning such that the label of information of inputted ground truth data matches the label of an output result.

For example, as illustrated in FIG. 4, the neural network performs learning by using, as a learning data set, N pieces of ground truth data in which an ultrasound image and a measurement item measurable in the image, of which the probability is 1 (100%), are one set.

For example, when the ultrasound image generated by image generation unit 14 is inputted to the neural network that has performed such learning, the probability for each measurement item is outputted for the inputted ultrasound image as illustrated in FIG. 3. In the example illustrated in FIG. 3, measurement A has a probability of 80% and can be estimated as a measurable measurement item candidate, and measurement C and measurement D have probabilities of 2% and 1%, respectively, and can be estimated as unmeasurable measurement item candidates.

As described above, measurement item estimation unit 161 estimates a measurement item candidate(s) that is/are measurable or unmeasurable. That is, measurement item estimation unit 161 can automatically estimate a measurable measurement item(s) as a measurement item candidate(s). Then, as illustrated in FIG. 2, display unit 15 displays a measurement item candidate(s) together with a display image in real time.

At this time, measurement item estimation unit 161 estimates a measurement item(s) having a predetermined probability or higher as measurable, and outputs the measurement item(s) as a measurement item candidate(s) to display unit 15. For example, as illustrated in FIG. 3, when it is assumed that the probabilities of measurement A, measurement B, measurement C, and measurement D are 80%, 17%, 2%, and 1%, respectively, and it is estimated that a measurement item having a probability of 80% or higher is measureable, measurement item estimation unit 161 outputs measurement A as a measurement item candidate to display unit 15. In this case, display unit 15 displays measurement A, which is a measurement item having a predetermined probability or higher, as a measurement item candidate.

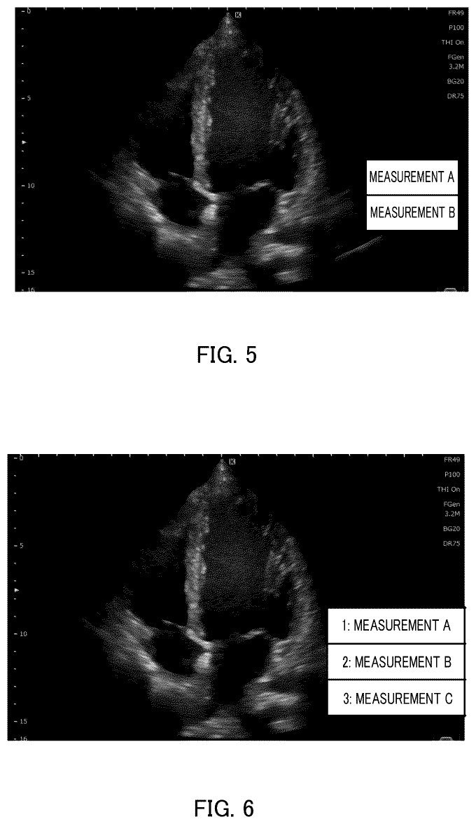

Further, in a case where there is a plurality of measurement items having a predetermined probability or higher, measurement item estimation unit 161 outputs the plurality of measurement items to display unit 15 as measurement item candidates. In this case, for example, as illustrated in FIG. 2, display unit 15 displays measurements A and B, which are measurement items having a predetermined probability or higher, as measurement item candidates. In this case, display unit 15 may display the measurement item candidates from left to right in descending order of probability as illustrated in FIG. 2, or may display the measurement item candidates from top to bottom in descending order of probability as illustrated in FIG. 5.

Further, as illustrated in FIG. 6, display unit 15 may display a predetermined number of measurement items in descending order of probability. The example illustrated in FIG. 6 represents a case where the predetermined number is three, and when it is assumed as illustrated in FIG. 3 that the probabilities of measurement A, measurement B, measurement C, and measurement D are 80%, 17%, 2%, and 1%, respectively, the top three measurements A, B, and C are displayed from top to bottom in descending order of probability. At this time, display unit 15 may display the order of the probability together with the measurement item.

Further, display unit 15 may display a predetermined number of measurement items having a predetermined probability or higher in descending order of probability.

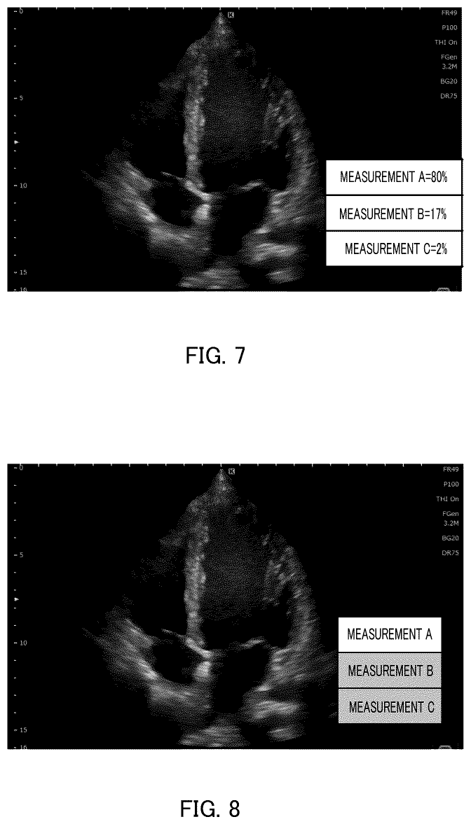

Further, as illustrated in FIG. 7, display unit 15 may display the probability of a measurement item together with the measurement item.

In addition, as illustrated in FIG. 8, display unit 15 may change the display aspect of a measurement item according to the probability of the measurement item, together with the measurement item. For example, it is configured according to the probability of a measurement item such that the color or the like of the area in which the measurement item is displayed is changed, or that the color or the like with which the measurement item itself is displayed is changed, and thus, the user can visually recognize a measurement item(s) having a high probability. For example, in the example illustrated in FIG. 8, the area in which measurement A having a high probability is displayed is bright, and areas in which measurements B and C having a low probability are displayed are dark, and thus, the user can visually recognize measurement A having a high probability.

As described above, display unit 15 displays a measurement item candidate(s) in real time such that the user can visually recognize a measurement item(s) with a high probability.

The user confirms measurement item candidate(s) displayed on display unit 15 and selects a desired measurement item. For example, in a case where the desired measurement item is measurement A as illustrated in FIG. 2, the user selects measurement A, for example, by using the control panel or the touch screen. An operation signal due to the selection is inputted to control unit 16 via operation input unit 11, and control unit 16 activates measurement unit 163. Specifically, control unit 16 activates measurement processing (e.g., a measurement program) of measurement A, which is included in measurement unit 163, and, while causing display unit 15 to display a display image in real time, causes measurement A with respect to the display image is performed in real time, and causes the measurement result to be displayed in real time.

Note that, in a case where measurement item estimation unit 161 is incapable of estimating a measurable measurement item candidate(s) or is capable of estimating only an unmeasurable measurement item candidate(s), display unit 15 displays unmeasurability, or displays that an ultrasound image is inappropriate for measurement.

As described above, in ultrasound diagnostic apparatus 1 according to the present embodiment, measurement item estimation unit 161 in control unit 16 automatically estimates a measurable measurement item candidate(s) by estimating a measurement item candidate(s), which is/are measurable or unmeasurable, and display unit 15 displays the measurement item candidate(s). Since display unit 15 displays the measurement item candidate(s) with respect to an observation area, the user can select a desired measurement item from the measurement item candidate(s), and control unit 16 can use measurement unit 163 to immediately measure the selected desired measurement item.

Currently, a plurality of automatic measurements utilizing an AI technology is mounted in a number of ultrasound diagnostic apparatuses. However, since organ/tissue recognition requires a large calculation load, it is difficult to simultaneously execute AI engines supporting a plurality of measurement items to perform automatic measurement in real time. In contrast, since the present embodiment utilizes an AI technology for measurement item candidate estimation, the calculation load is small and the automatic measurement can be performed in real time as compared with a case where AI engines supporting a plurality of measurement items are simultaneously executed.

In addition, in operation input unit 11, the number of operation buttons is limited, and in the touch screen display, the number of operation buttons that can be displayed on display unit 15 is limited. Therefore, even when a desired automatic measurement is intended to be executed from among a plurality of mounted automatic measurements by an operation of a button, the automatic measurement cannot be immediately executed, which results in an increase in an inspection time and an increase in a load on a patient and/or the user. In contrast, in the present embodiment, measurement item estimation unit 161 automatically estimates a measurable measurement item candidate(s), and display unit 15 displays the measurement item candidate(s). Therefore, when the user selects a desired measurement item from a displayed measurement item candidate(s), automatic measurement can be immediately performed. As a result, it is possible to shorten an inspection time and reduce a load on a patient and/or the user.

Further, in the related art, a desired measurement item is searched for from a menu in which a number of measurement items are defined for each observation region. In contrast, in the present embodiment, measurement item estimation unit 161 automatically estimates a measurable measurement item candidate(s), and display unit 15 displays the measurement item candidate(s). Therefore, it is not necessary to search for a desired measurement item, it is possible to shorten an inspection time, and it is possible to reduce a load on a patient and/or the user.

### <Variation 1>

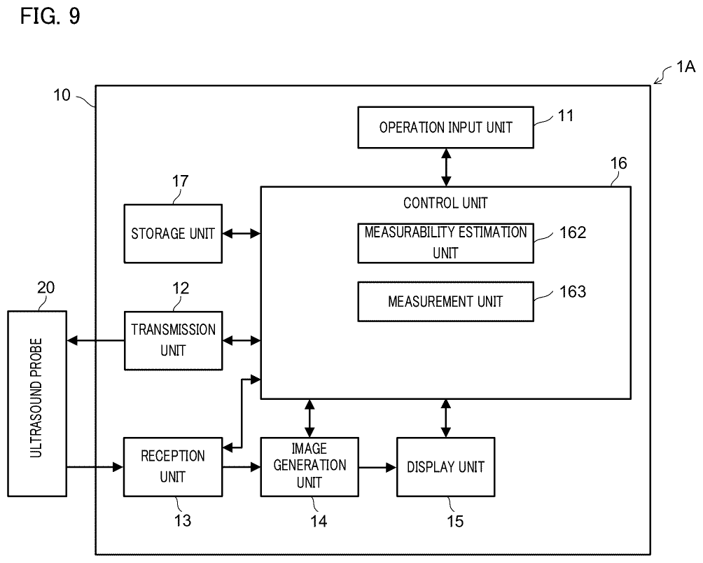

FIG. 9 is a block diagram illustrating another example of a main part of ultrasound diagnostic apparatus 1A according to the embodiment of the present invention.

In this variation, ultrasound diagnostic apparatus 1A has the same configuration as that of ultrasound diagnostic apparatus 1 illustrated in FIG. 1 except for measurability estimation unit 162. Therefore, for ultrasound diagnostic apparatus 1A, a description of a configuration overlapping with that of ultrasound diagnostic apparatus 1 will be omitted.

In the present variation, ultrasound diagnostic apparatus 1A includes measurability estimation unit 162 instead of measurement item estimation unit 161 in ultrasound diagnostic apparatus 1 illustrated in FIG. 1. Further, in the present variation, operation input unit 11 functions as a designation unit that designates a measurement item.

Measurability estimation unit 162 (the estimation unit in the present invention) estimates whether a measurement item specified using operation input unit 11 is measurable in an observation area. That is, unlike the present embodiment described above, measurability estimation unit 162 does not estimate any measurement item candidate, but estimates whether a designated measurement item(s) is/are measurable.

The estimation in measurability estimation unit 162 is the same as that in measurement item estimation unit 161 described above, and the estimation is performed using an AI technology. Specifically, as an example, measurability estimation unit 162 is also a learned model or a discriminator using a neural network in the same manner as measurement item estimation unit 161. Further, in the same manner as measurement item estimation unit 161, the learned model or the discriminator is also a model learned by associating an ultrasound image (cross-sectional image) with a measurement item candidate (scc FIG. 3 and FIG. 4).

Then, in the present variation, measurability estimation unit 162 in control unit 16 estimates whether a measurement item(s) designated by the user (desired by the user) is/are measurable in an observation area, and display unit 15 displays information on whether the designated measurement item(s) is/are measurable. Since display unit 15 displays information on whether a designated measurement item(s) is/are measurable, the user can select a desired measurement item based on the information, and control unit 16 can use measurement unit 163 to measure the selected measurement item.

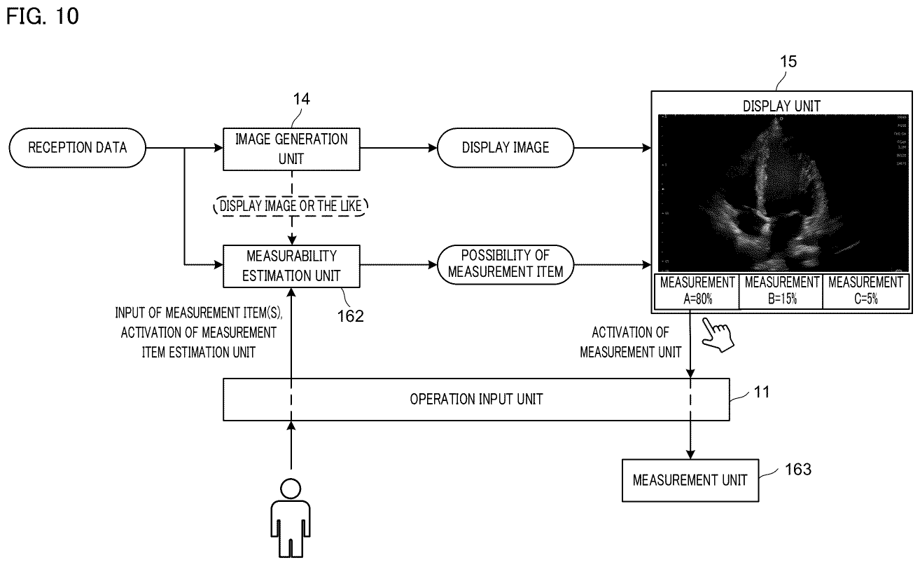

Here, display and selection of information on a measurement item and measurement of the selected measurement item in the present variation will be described with reference to FIG. 10. FIG. 10 is a block diagram provided for describing an example of an information display method of ultrasound diagnostic apparatus 1A illustrated in FIG. 9.

Even in ultrasound diagnostic apparatus 1A, reception data acquired by using ultrasound probe 20, transmission unit 12, and reception unit 13 is inputted to image generation unit 14. The inputted reception data is generated into ultrasound image data or a display image by image generation unit 14 and is outputted, as a display image, for example, a cross-sectional image to display unit 15, and display unit 15 displays the display image in real time.

At this time, measurability estimation unit 162 is activated by an operation of the user, and at least one measurement item is inputted (designated) by the user. Measurability estimation unit 162 estimates whether the designated measurement item(s) is/are measurable in an observation area, by using the reception data from reception unit 13 as input data. Since the processing here is also performed in real time according to the display image, it is desirable to estimate whether the designated measurement item(s) is/are measurable, by using the reception data. Note that, it may be estimated whether the designated measurement item(s) is/are measurable, by using ultrasound image data and/or a display image generated by image generation unit 14, depending on the processing capability of control unit 16 (in a case where the processing capability thereof is high). Information on whether the measurement item(s) is/are measurable, for example, probability information, which is estimated by measurement item estimation unit 161, is outputted to display unit 15, and display unit 15 displays the measurement item(s) and the probability information in real time.

Display unit 15 also displays a measurement item(s) and probability information together with a display image in real time. For example, as illustrated in FIG. 10, it is assumed that three measurement items “measurement A”, “measurement B”, and “measurement C” are designated and displayed on display unit 15. In this case, display unit 15 also displays the probability information together with the measurement items.

The user selects a desired measurement item from among the designated measurement items with reference to the probability information together with the measurement items, and thus, measurement unit 163 is activated, and measurement of the selected measurement item is executed. For example, when the user selects “measurement A”, measurement unit 163 is activated, and measurement of the selected “measurement A” is executed.

In the estimation of whether a designated measurement item(s) is/are measurable, the measurement item(s) and the probability thereof are acquired as probability information by using the learned model using the neural network described above.

As described above, measurability estimation unit 162 estimates whether a designated measurement item(s) is/are measurable. Then, as illustrated in FIG. 10, display unit 15 displays, together with a display image and a measurement item(s), the probability indicating the measurability of the measurement item(s) in real time.

The user confirms the measurement item(s) and the probability indicating the measurability of the measurement item(s), which are displayed on display unit 15, and selects a desired measurement item. For example, in a case where the desired measurement item is measurement A as illustrated in FIG. 10, the user selects measurement A, for example, by using the control panel or the touch screen. An operation signal due to the selection is inputted to control unit 16 via operation input unit 11, and control unit 16 activates measurement unit 163. Specifically, control unit 16 activates measurement processing (e.g., a measurement program) of measurement A, which is included in measurement unit 163, and, while causing display unit 15 to display a display image in real time, causes measurement A with respect to the display image to be performed in real time, and causes the measurement result to be displayed in real time.

Note that, in a case where measurability estimation unit 162 estimates that a designated measurement item(s) is/are unmeasurable, measurability estimation unit 162 causes display unit 15 to display immeasurability, or display that an ultrasound image is inappropriate for measurement.

As described above, in ultrasound diagnostic apparatus 1A of the present variation, measurability estimation unit 162 in control unit 16 estimates whether a measurement item(s) designated by the user is/are measurable in an observation area, and display unit 15 displays information on whether the designated measurement item(s) is/are measurable. Since display unit 15 displays a measurement item(s) and information on whether the measurement item(s) is/are measurable, the user can select a desired measurement item from the measurement item(s), and control unit 16 can use measurement unit 163 to immediately measure the selected desired measurement item.

In addition, since ultrasound diagnostic apparatus 1A of the present variation also utilizes an AI technology to estimate whether a measurement item(s) is/are measurable, the calculation load is small and the automatic measurement can be performed in real time as compared with a case where AI engines supporting a plurality of measurement items are simultaneously executed.

Further, even in ultrasound diagnostic apparatus 1A according to the present variation, measurability estimation unit 162 estimates whether a designated measurement item(s) is/are measurable, and display unit 15 displays information on whether the designated measurement item(s) is/are measurable. For this reason, the user can immediately perform automatic measurement by selecting a desired measurement item from the displayed measurement item(s). As a result, it is possible to shorten an inspection time and reduce a load on a patient and/or the user.

Further, even in ultrasound diagnostic apparatus 1A according to the present variation, measurability estimation unit 162 estimates whether a designated measurement item(s) is/are measurable, and display unit 15 displays information on whether the designated measurement item(s) is/are measurable. For this reason, it is not necessary to search for a desired measurement item, it is possible to shorten an inspection time, and it is possible to reduce a load on a patient and/or the user.

Note that, the following supplementary notes will be further disclosed with respect to the above description.

### (Supplementary Note 1)

An ultrasound diagnostic apparatus, comprising: an ultrasound probe that transmits and receives ultrasound to and from a subject; a display; and at least one hardware processor, wherein the at least one hardware processor generates ultrasound image data based on a reception signal obtained from an observation area, designates a measurement item, estimates whether the measurement item having been designated is measurable, and causes the display to display information on whether the measurement item having been designated is measurable.

### (Supplementary Note 2)

The ultrasound diagnostic apparatus according to Supplementary Note 1, wherein the at least one hardware processor includes a discriminator that uses a neural network and is learned by associating an ultrasound image with the measurement item.

### (Supplementary Note 3)

The ultrasound diagnostic apparatus according to Supplementary Note 1, wherein in a case where it has been estimated that the measurement item having been designated is unmeasurable, the at least one hardware processor causes immeasurability to be displayed, or causes displaying that an ultrasound image is inappropriate for the measurement.

### (Supplementary Note 4)

The ultrasound diagnostic apparatus according to Supplementary Note 1, further comprising a measurer that makes the measurement item in the observation area measurable even in a case where it has been estimated that the measurement item having been designated is unmeasurable.

### (Supplementary Note 5)

A non-transitory computer-readable recording medium storing an ultrasound diagnostic program, the ultrasound diagnostic program causing a computer to execute: transmitting and receiving, by an ultrasound probe, ultrasound to and from a subject; generating ultrasound image data based on a reception signal obtained from an observation area; estimating a measurement item candidate that is measurable or unmeasurable; and displaying the measurement item candidate.

### (Supplementary Note 6)

The non-transitory computer-readable recording medium according to Supplementary Note 5, wherein in the displaying, the measurement item candidate is displayed in real time.

### (Supplementary Note 7)

The non-transitory computer-readable recording medium according to Supplementary Note 5, wherein: in the generating, the ultrasound image data is generated in real time, and in the estimating, the measurement item candidate is estimated in real time with respect to the ultrasound image data generated in the real time.

### (Supplementary Note 8)

The non-transitory computer-readable recording medium according to Supplementary Note 5, wherein in the estimating, a probability of whether the measurement item candidate is measurable is estimated by using the ultrasound image data as input data.

### (Supplementary Note 9)

The non-transitory computer-readable recording medium according to Supplementary Note 8, wherein in the displaying, one or a plurality of the measurement item candidates having the probability equal to or greater than a predetermined probability or a predetermined number of a plurality of the measurement item candidates in descending order of the probability is displayed, or a predetermined number of a plurality of the measurement item candidates in descending order of the probability is displayed, the plurality of measurement item candidates having the probability equal to or greater than a predetermined probability.

### (Supplementary Note 10)

The non-transitory computer-readable recording medium according to Supplementary Note 5, wherein the ultrasound image data is data on at least one of a B-mode image, a Doppler image, an M-mode image, and/or an elastography image, and the Doppler image includes a color Doppler image, a pulse Doppler image, a continuous wave Doppler image, and a tissue Doppler image.

### (Supplementary Note 11)

The non-transitory computer-readable recording medium according to Supplementary Note 5, wherein in the displaying, a touch input for selecting the measurement item candidate having been displayed is detected by using a touch screen.

### (Supplementary Note 12)

The non-transitory computer-readable recording medium according to Supplementary Note 5, wherein in the displaying, an input for selecting the measurement item candidate having been displayed is detected by a control panel including a key.

### (Supplementary Note 13)

The non-transitory computer-readable recording medium according to Supplementary Note 5, wherein in the estimating, processing is performed with a learned model using a neural network.

### (Supplementary Note 14)

The non-transitory computer-readable recording medium according to Supplementary Note 13, wherein the learned model is a model learned by associating an ultrasound image with the measurement item candidate.

### (Supplementary Note 15)

The non-transitory computer-readable recording medium according to Supplementary Note 5, wherein in the estimating, in a case where a measurement item candidate that is measurable is inestimable or in a case where only a measurement item candidate that is unmeasurable is estimable, unmeasurability is displayed, or it is displayed that an ultrasound image is inappropriate for measurement.

### (Supplementary Note 16)

The non-transitory computer-readable recording medium according to Supplementary Note 5, further causing the computer to execute measuring the measurement item in the observation area even in a case where a measurement item candidate that is measurable is inestimable by the measurement item estimating or even in a case where only a measurement item candidate that is unmeasurable is estimable.

### (Supplementary Note 17)

A non-transitory computer-readable recording medium storing an ultrasound diagnostic program, the ultrasound diagnostic program causing a computer to execute: transmitting and receiving, by an ultrasound probe, ultrasound to and from a subject; generating ultrasound image data based on a reception signal obtained from an observation area; designating a measurement item; estimating whether the measurement item having been designated is measurable; and displaying, by a display, information on whether the measurement item having been designated is measurable.

### (Supplementary Note 18)

The recording medium according to Supplementary Note 17, wherein in the displaying, the information on whether the measurement item having been designated is measurable is displayed in real time.

### (Supplementary Note 19)

The non-transitory computer-readable recording medium according to Supplementary Note 17, wherein in the generating, the ultrasound image data is generated in real time, and in the estimating, it is estimated, with respect to the ultrasound image data generated in the real time, whether the designated measurement item is measurable, in real time.

### (Supplementary Note 20)

The non-transitory computer-readable recording medium according to Supplementary Note 17, wherein in the estimating, a probability of whether the measurement item having been designated is measurable is estimated by using the ultrasound image data as input data.

### (Supplementary Note 21)

The non-transitory computer-readable recording medium according to Supplementary Note 20, wherein in the displaying, as the information on whether the measurement item having been designated is measurable, one or a plurality of the measurement items having the probability equal to or greater than a predetermined probability or a predetermined number of a plurality of the measurement items in descending order of the probability is displayed, or, as the information on whether the measurement item having been designated is measurable, a predetermined number of a plurality of the measurement items in descending order of the probability is displayed, the plurality of measurement items having the probability equal to or greater than a predetermined probability.

### (Supplementary Note 22)

The non-transitory computer-readable recording medium according to Supplementary Note 17, wherein the measurement item includes at least one of measurement items related to a B-mode image, a Doppler image, an M-mode image, and/or an elastography image, and the Doppler image includes a color Doppler image, a pulse Doppler image, a continuous wave Doppler image, and a tissue Doppler image.

### (Supplementary Note 23)

The non-transitory computer-readable recording medium according to Supplementary Note 17, wherein in the estimating, processing is performed by a discriminator learned by associating an ultrasound image with the measurement item by a neural network.

### (Supplementary Note 24)

The non-transitory computer-readable recording medium according to Supplementary Note 17, wherein in the estimating, in a case where it has been estimated that the measurement item having been designated is unmeasurable, immeasurability is displayed, or it is displayed that an ultrasound image is inappropriate for the measurement.

### (Supplementary Note 25)

The non-transitory computer-readable recording medium according to Supplementary Note 17, further causing the computer to execute measuring of making the measurement item in the observation area measurable, even in a case where it has been estimated in the estimating that the measurement item having been designated is unmeasurable.

### (Supplementary Note 26)

An information display method of an ultrasound diagnostic apparatus, comprising: transmitting and receiving, by an ultrasound probe, ultrasound to and from a subject; generating ultrasound image data based on a reception signal obtained from an observation area; designating a measurement item; estimating whether the measurement item having been designated is measurable; and displaying, by a display, information on whether the measurement item having been designated is measurable.

Any of the embodiment described above is only illustration of an exemplary embodiment for implementing the present invention, and the technical scope of the present invention shall not be construed limitedly thereby. That is, the present invention can be implemented in various forms without departing from the gist or the main features thereof.

For example, in measurement item estimation unit 161 and measurability estimation unit 162, the neural network is used as an exemplary AI technology, but any other AI technology may be used.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic apparatus, comprising:
an ultrasound probe that transmits and receives ultrasound to and from a subject;
a display; and
at least one hardware processor, wherein
the at least one hardware processor
generates ultrasound image data based on a reception signal obtained from an observation area,
estimates a measurement item candidate that is measurable or unmeasurable, and
causes the display to display the measurement item candidate.

2. The ultrasound diagnostic apparatus according to claim 1, wherein
the display displays the measurement item candidate in real time.

3. The ultrasound diagnostic apparatus according to claim 1, wherein
the at least one hardware processor
generates the ultrasound image data in real time, and
estimates, with respect to the ultrasound image data generated in the real time, the measurement item candidate in real time.

4. The ultrasound diagnostic apparatus according to claim 1, wherein
the at least one hardware processor estimates a probability of whether the measurement item candidate is measurable, by using the ultrasound image data as input data.

5. The ultrasound diagnostic apparatus according to claim 4, wherein:
the display displays
one or a plurality of the measurement item candidates having the probability equal to or greater than a predetermined probability or a predetermined number of a plurality of the measurement item candidates in descending order of the probability, or
a predetermined number of a plurality of the measurement item candidates in descending order of the probability, the plurality of measurement item candidates having the probability equal to or greater than a predetermined probability.

6. The ultrasound diagnostic apparatus according to claim 1, wherein
the ultrasound image data is data on at least one of a B-mode image, a Doppler image, an M-mode image, and/or an elastography image, and
the Doppler image includes a color Doppler image, a pulse Doppler image, a continuous wave Doppler image, and a tissue Doppler image.

7. The ultrasound diagnostic apparatus according to claim 1, wherein
the display includes a touch screen and detects a touch input for selecting the measurement item candidate having been displayed.

8. The ultrasound diagnostic apparatus according to claim 1, further comprising a control panel that includes a key for selecting the measurement item candidate displayed at the display.

9. The ultrasound diagnostic apparatus according to claim 1, wherein
the at least one hardware processor includes a learned model using a neural network.

10. The ultrasound diagnostic apparatus according to claim 9, wherein
the learned model is a model learned by associating an ultrasound image with the measurement item candidate.

11. The ultrasound diagnostic apparatus according to claim 1, wherein
in a case where the at least one hardware processor is incapable of estimating the measurement item candidate, which is measureable, or is capable of estimating only the measurement item candidate, which is unmeasurable, the at least one hardware processor causes unmeasurability to be displayed or causes displaying that an ultrasound image is inappropriate for measurement.

12. The ultrasound diagnostic apparatus according to claim 1, further comprising a measurer that measures the measurement item in the observation area even in a case where the measurement item candidate, which is measurable, is inestimable or even in a case where only the measurement item candidate, which is unmeasurable, is estimable.

13. An ultrasound diagnostic apparatus, comprising:
an ultrasound probe that transmits and receives ultrasound to and from a subject;
a display; and
at least one hardware processor, wherein
the at least one hardware processor
generates ultrasound image data based on a reception signal obtained from an observation area,
designates a measurement item,
estimates whether the measurement item having been designated is measurable, and
causes the display to display information on whether the measurement item having been designated is measurable.

14. The ultrasound diagnostic apparatus according to claim 13, wherein
the display displays the information on whether the measurement item having been designated is measurable, in real time.

15. The ultrasound diagnostic apparatus according to claim 13, wherein:
the at least one hardware processor
generates the ultrasound image data in real time, and
estimates, with respect to the ultrasound image data generated in the real time, whether the measurement item having been designated is measurable, in real time.

16. The ultrasound diagnostic apparatus according to claim 13, wherein
the at least one hardware processor estimates a probability of whether the measurement item having been designated is measurable, by using the ultrasound image data as input data.

17. The ultrasound diagnostic apparatus according to claim 16, wherein:
the display displays, as the information on whether the measurement item having been designated is measurable,
one or a plurality of the measurement items having the probability equal to or greater than a predetermined probability or a predetermined number of a plurality of the measurement items in descending order of the probability, or
a predetermined number of a plurality of the measurement items in descending order of the probability, the plurality of measurement items having the probability equal to or greater than a predetermined probability.

18. The ultrasound diagnostic apparatus according to claim 13, wherein
the measurement item includes at least one of measurement items related to a B-mode image, a Doppler image, an M-mode image, and/or an elastography image, and
the Doppler image includes a color Doppler image, a pulse Doppler image, a continuous wave Doppler image, and a tissue Doppler image.

19. A non-transitory computer-readable recording medium storing an ultrasound diagnostic program, the ultrasound diagnostic program causing a computer to execute:
transmitting and receiving, by an ultrasound probe, ultrasound to and from a subject;
generating ultrasound image data based on a reception signal obtained from an observation area;
estimating a measurement item candidate that is measurable or unmeasurable; and
displaying the measurement item candidate.

20. An information display method of an ultrasound diagnostic apparatus, the information display method comprising:
transmitting and receiving, by an ultrasound probe, ultrasound to and from a subject;
generating ultrasound image data based on a reception signal obtained from an observation area;
estimating a measurement item candidate that is measurable or unmeasurable; and
displaying, by a display, the measurement item candidate.
